(19) United States
(12) Patent Application Publication
(10) Pub. No.: US 2022/0203076 A1
(43) Pub. Date: Jun. 30, 2022

(54) OPERATION DEVICE, AND REMOTE OPERATION SYSTEM FOR ELONGATED BODY

(71) Applicant: TERUMO KABUSHIKI KAISHA, Tokyo (JP)

(72) Inventors: Shuhei MATSUSHITA, Hadano-city (JP); Yuki SAKAGUCHI, Isehara-city (JP); Tomoya KOMATSU, Hadano-city (JP); Eisuke FURUICHI, Hadano-city (JP)

(73) Assignee: TERUMO KABUSHIKI KAISHA, Tokyo (JP)

(21) Appl. No.: 17/694,752

(22) Filed: Mar. 15, 2022

Related U.S. Application Data
(63) Continuation of application No. PCT/JP2020/031176, filed on Aug. 18, 2020.

(30) Foreign Application Priority Data
Sep. 26, 2019 (JP) 2019-176147

(51) Int. Cl. A61M 25/09; A61M 25/01; A61M 25/04

(52) U.S. Cl. CPC A61M 25/09041; A61M 2025/0293; A61M 25/04; A61M 25/0105

(57) ABSTRACT

An operation device is configured to remotely operate a medical device including a movement mechanism configured to move an elongated body to be inserted into a living body forward and backward along the longitudinal direction of the elongated body, and includes an endless linear member. The medical device is operated based on a forward and backward movement of the linear member in the endless axis direction along an endless axis.

### CROSS-REFERENCES TO RELATED APPLICATIONS

This application is a continuation of International Patent Application No. PCT/JP2020/031176 filed on Aug. 18, 2020, which claims priority to Japanese Patent Application No. 2019-176147, the entire content of both of which is incorporated herein by reference.

### TECHNOLOGICAL FIELD

The present disclosure relates to an operation device and a remote operation system for an elongated body.

### BACKGROUND DISCUSSION

In recent years, remote operation systems that remotely operate a catheter inserted into a blood vessel have been known. Using a remote operation system, a health care worker can be prevented from being exposed to radiation during a medical procedure. JP-T-2011-519678 discloses a robotic vascular catheter system including a bedside system and a workstation as one type of remote operation system. In the robotic vascular catheter system described in this Japanese application publication, the bedside system can be remotely operated via the workstation. The workstation disclosed in this Japanese application publication includes a user interface including a joystick and jog buttons.

### SUMMARY

In the robotic vascular catheter system described in JP-T-2011-519678, a movement amount of a catheter held in the bedside system is controlled by operating the joystick and the jog buttons of the workstation. Therefore, an operation performed by the workstation serving as the operation device described in JP-T-2011-519678 is greatly different from an actual catheter operation performed by a health care worker. Therefore, in the robotic vascular catheter system described in JP-T-2011-519678, there is room for further improvement in terms of operability for the health care worker.

The operation device and remote operation system disclosed here are capable of improving operability for a health care worker.

An operation device according to a first aspect is an operation device configured to remotely operate a medical device that includes a movement mechanism configured to move an elongated medical body that is insertable into a living body forward and backward along a longitudinal direction of the elongated medical body. The operation device comprises: an endless linear member operatively connectable to the medical device and operable by a user to undergo forward and backward movement in an endless axial direction along an endless axis. The elongated medical body is operated based on the forward and backward movement of the linear member in the endless axial direction along the endless axis.

An operation device according to an embodiment of the present disclosure further includes: a forward and backward movement detection sensor capable of detecting a forward and backward movement of the linear member in the endless axis direction; and a control device configured to transmit, to the medical device, forward and backward movement information related to a forward and backward movement of the linear member in the endless axis direction, the forward and backward movement being detected by the forward and backward movement detection sensor.

An operation device according to an embodiment of the present disclosure further includes a plurality of support members that are wound around by the linear member and that support the linear member.

According to an embodiment of the present disclosure, at least one of the plurality of support members is a rotatable member that rotates around a central axis according to the forward and backward movement of the linear member in the endless axis direction.

An operation device according to an embodiment of the present disclosure includes a variable resistance mechanism capable of changing a rotatable resistance of the rotatable member around the central axis. The control device is configured to control the variable resistance mechanism based on a load resistance received by the elongated body.

According to an embodiment of the present disclosure, the rotatable member includes: a rotatable main body that rotates around the central axis; and a plurality of rotatable bearing bodies that are attached on an outer surface of the rotatable main body around the center axis, and that rotate according to rotational movement of the linear member around the endless axis while being in contact with the linear member. The plurality of rotatable bearing bodies form a first rotatable bearing body group and a second rotatable bearing body group that are disposed at different positions in a central axis direction along the central axis at intervals around the central axis, and the linear member is in contact with and supported by rotatable bearing bodies belonging to the first rotatable bearing body group and rotatable bearing bodies belonging to the second rotatable bearing body group at a position between the first rotatable bearing body group and the second rotatable bearing body group in the central axis direction.

An operation device according to an embodiment of the present disclosure includes a variable bearing resistance mechanism capable of changing a rotatable resistance of the rotatable bearing body with respect to the rotatable main body. The control device is configured to control the variable bearing resistance mechanism based on a load resistance received by the elongated body.

According to an embodiment of the present disclosure, the movement mechanism is capable of rotating the elongated body around a central axis of the elongated body, a rotation detection sensor capable of detecting rotation of the linear member in an axial rotational direction around the endless axis is provided, and the control device is configured to transmit, to the medical device, rotation information related to rotational movement of the linear member in the axial rotational direction, the rotational movement being detected by the rotation detection sensor.

In accordance with another aspect, an operation device is configured to remotely operate a movement mechanism that is configured to axially move an elongated medical body in a forward direction and in a backward direction while the elongated medical body is positioned in a living body. The operation device comprises: an endless elongated member that includes a central axis and that is operable by a user to move the endless elongated member in one direction along the central axis of the endless elongated member and in an opposite direction along the central axis of the endless elongated member; a movement detection sensor positioned and configured to detect the movement of the endless elongated member in the one direction and in the opposite direction; and a control device that receives input from the movement detection sensor about the movement of the endless elongated member in the one direction and in the opposite direction and that is operatively connectable to the movement mechanism to control the movement mechanism and move the elongated medical body in the forward direction in response to the movement of the endless elongated member in the one direction as detected by the movement detection sensor and to move the elongated medical body in the backward direction in response to the movement of the endless elongated member in the opposite direction as detected by the movement detection sensor.

Another aspect involves a method comprising: manually moving an endless elongated member that includes a central axis to move the endless elongated member in one direction along the central axis of the endless elongated member; and operating a movement mechanism that is remotely located relative to the endless elongated member and that is operatively connected to an elongated medical device positioned in a living body based on the movement of the endless elongated member in the one direction along the central axis of the endless elongated member to advance the elongated medical device in the living body.

A remote operation system according to another aspect of the present disclosure includes the above-mentioned operation device and the medical device that is remotely operated by the operation device.

The operation device and remote operation system disclosed here provide improved operability for a health care worker.

### BRIEF DESCRIPTION OF THE DRAWINGS

FIG. 1 is a diagram illustrating a remote operation system according to an embodiment of the present disclosure.

FIG. 2 is a diagram illustrating a configuration example of a medical device illustrated in FIG. 1.

FIG. 3 is a diagram illustrating a configuration example of the operation device illustrated in FIG. 1.

FIG. 4 is a diagram illustrating details of a variable resistance mechanism and a variable bearing resistance mechanism that act on one support member illustrated in FIG. 3.

FIG. 5 is a cross-sectional view taken along the section line V-V in FIG. 4.

### DETAILED DESCRIPTION

Hereinafter, embodiments of an operation device and a remote operation system representing examples of the operation device and remote operation system disclosed here will be described with reference to the drawings. In the drawings, common members and portions are denoted by the same reference numerals.

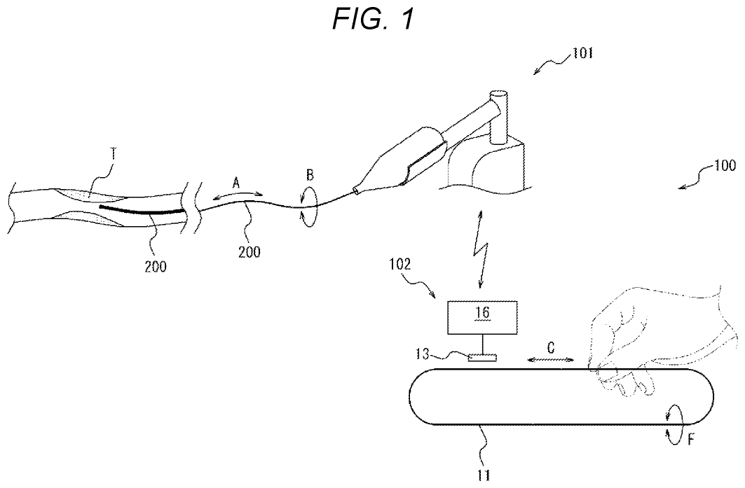

FIG. 1 is a diagram illustrating a remote operation system 100 according to one embodiment. As shown in FIG. 1, the remote operation system 100 includes a medical device 101, and an operation device 102 according to an embodiment of the present disclosure. In addition to the medical device 101 and the operation device 102, the remote operation system 100 may include, for example, another device capable of communicating with at least one of the medical device 101 and the operation device 102 in a wired or wireless manner. Hereinafter, as an example of the remote operation system 100, a configuration including the medical device 101 and the operation device 102 will be described.

The remote operation system 100 according to the present embodiment is used in, for example, a vascular catheter surgery in which a medical elongated body or elongated medical device 200 (hereinafter, referred to as the “elongated body 200”) such as a guide wire or a catheter is inserted into a blood vessel of a patient to perform a predetermined medical procedure on a lesion area T. In the vascular catheter surgery, the medical device 101 is disposed in the vicinity of the patient into whom the elongated body 200 is to be inserted. The medical device 101 includes a movement mechanism 110 that moves the elongated body 200 to be inserted into a living body forward and backward along a longitudinal direction A of the elongated body 200. First, the elongated body 200 is inserted into the blood vessel of the patient by, for example, a manual operation of a health care worker himself/herself. Then, a portion of the elongated body 200 extending to the outside of the living body of the patient is set in the medical device 101 by the health care worker. The medical device 101 and the operation device 102 can communicate with each other in a wired or wireless manner. The medical device 101 can be remotely operated by the operation device 102. The operation device 102 is disposed at a position away from a position where the patient is present. The position where the operation device 102 is disposed may be in an operating room in which surgery is performed, or may be located at another position outside the operating room. The health care worker can remotely operate the medical device 101 through the operation device 102. That is, the health care worker can move the elongated body 200 held by the medical device 101 forward and backward by operating the operation device 102 and operating the movement mechanism 110 of the medical device 101.

Hereinafter, details of the remote operation system 100 according to the present embodiment will be described.

<Medical Device 101>

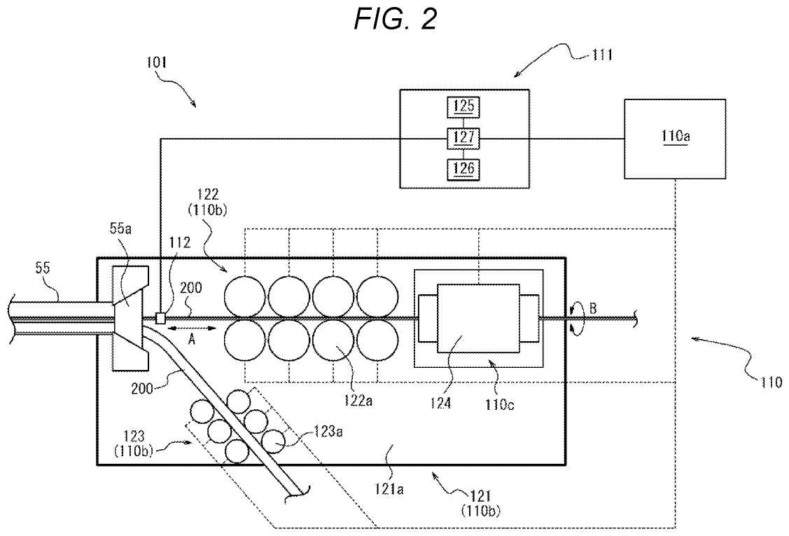

FIG. 2 is a diagram illustrating an example of the configuration of the medical device 101. As shown in FIG. 2, the medical device 101 according to the present embodiment includes the movement mechanism 110, a mechanism control device 111, and a load sensor 112.

As described above, the movement mechanism 110 can move the elongated body 200 to be inserted into a living body forward and backward along the longitudinal direction A. Further, the movement mechanism 110 according to the present embodiment can rotate the elongated body 200 around a central axis of the elongated body 200 in addition to the forward and backward movement.

The movement mechanism 110 according to the present embodiment includes a drive source 110a, a forward and backward moving mechanism 110b that is driven by the drive source 110a to move the elongated body 200 forward and backward, and a rotating mechanism 110c that is driven by the drive source 110a to rotate the elongated body 200 around the central axis.

The drive source 110a is implemented by, for example, an electric motor, but is not particularly limited as long as the drive source 110a can drive the forward and backward moving mechanism 110b and the rotating mechanism 110c. Driving of the drive source 110a is controlled by the mechanism control device 111.

The forward and backward moving mechanism 110b includes a support main body 121, a first rotatable body group 122, and a second rotatable body group 123.

The support main body 121 supports the elongated body 200. In the support main body 121, another guide tube 55 that guides the elongated body 200 to be moved forward and backward by the forward and backward moving mechanism 110b to the lesion area T (see FIG. 1) may be attached in advance. In FIG. 2, a connector 55a provided at a proximal end portion of the guide tube 55 is attached to the support main body 121. The elongated body 200 supported by the support main body 121 is introduced from the connector 55a to the lesion area T in the living body through the guide tube 55.

The first rotatable body group 122 includes a plurality of rotatable bodies 122a. The plurality of rotatable bodies 122a constituting the first rotatable body group 122 are rotatably attached to the support main body 121. The rotatable bodies 122a rotate with respect to the support main body 121 by a driving force of the drive source 110a. More specifically, the rotatable bodies 122a rotate around respective rotation axes extending in a direction orthogonal to a support surface 121a of the support main body 121 that supports the elongated body 200. The plurality of rotatable bodies 122a of the first rotatable body group 122 can move the elongated body 200 forward and backward in the longitudinal direction A by rotating while sandwiching the elongated body 200 supported by the support surface 121a of the support main body 121 between the rotatable bodies 122a.

The second rotatable body group 123 is similar to the first rotatable body group 122, but the positions where the rotatable body groups 122, 123 are attached to the support main body 121 are different. The forward and backward moving mechanism 110b illustrated in FIG. 2 includes the first rotatable body group 122 and the second rotatable body group 123, but may include only the first rotatable body group 122. However, as in the present embodiment, by providing the second rotatable body group 123 separately from the first rotatable body group 122, two different elongated bodies 200 can be set at the same time. Therefore, the two elongated bodies 200 can be alternately moved forward and backward without exchanging the two elongated bodies 200. For example, a guide wire serving as one elongated body 200 can be moved forward and backward by the first rotatable body group 122, and a balloon catheter serving as the other elongated body 200 can be moved forward and backward by the second rotatable body group 123. The second rotatable body group 123 includes a plurality of rotatable bodies 123a. Since the configuration of the second rotatable body group 123 is similar to that of the above-described first rotatable body group 122, a detailed description of the configuration of the second rotatable body group 123 is not repeated.

The rotating mechanism 110c is driven by the drive source 110a to rotate the elongated body 200 around the central axis of the elongated body 200. Specifically, the rotating mechanism 110c is supported by the support main body 121 of the forward and backward moving mechanism 110b, and can rotate the one elongated body 200, which can be moved forward and backward by the first rotatable body group 122, around the central axis of the elongated body 200 along the longitudinal direction A. The rotating mechanism 110c shown in FIG. 2 includes a rotatable body 124 that is rotatable with respect to the support main body 121 of the forward and backward moving mechanism 110b. The rotatable body 124 rotates with respect to the support main body 121 by a driving force of the drive source 110a. For example, the rotatable body 124 rotates around the central axis of the elongated body 200 in a state in which an outer circumferential surface of the elongated body 200 is sandwiched from the outside in a radial direction. Accordingly, as the rotatable body 124 rotates, the elongated body 200 sandwiched by the rotating body 124 also rotates around the central axis. That is, the rotatable body 124 is a co-rotating body that rotates around the central axis of the elongated body 200 together with the elongated body 200. However, the rotatable body 124 may rotate together with the elongated body 200 around the central axis of the elongated body 200, and the rotatable body 124 may hold the elongated body 200 by a method other than sandwiching. Although the rotatable body 124 of the rotating mechanism 110c shown in FIG. 2 is supported by the support main body 121 of the forward and backward moving mechanism 110b, the rotatable body 124 may be supported by another member.

The mechanism control device 111 controls an operation of the movement mechanism 110. Specifically, the mechanism control device 111 according to the present embodiment controls driving of the drive source 110a.

The mechanism control device 111 according to the present embodiment includes a communication unit 125, a storage unit 126, and a control unit 127.

The communication unit 125 can communicate with the operation device 102 in a wired or wireless manner. The communication unit 125 is electrically connected to a communication unit 25 in a control device 16 of the operation device 102 by, for example, an electric signal line capable of transmitting and receiving an electric signal. The communication unit 125 can receive, from the operation device 102, forward and backward movement information and rotation information of a linear member 11 (see FIG. 1) to be described later. The communication unit 125 can transmit, to the operation device 102, resistance information related to a load resistance received by the elongated body 200 and detected by a load sensor 112 to be described later.

The storage unit 126 stores a processing result from the control unit 127. The storage unit 126 may store various programs to be executed by the control unit 127. The storage unit 126 can be implemented by, for example, a random access memory (RAM), a read only memory (ROM), or the like.

The control unit 127 controls operations of the communication unit 125 and the storage unit 126. The control unit 127 processes various types of information received from the communication unit 125 and the storage unit 126.

The control unit 127 controls driving of the drive source 110a. Further, the control unit 127 controls an operation of the movement mechanism 110. Specifically, the control unit 127 controls, based on the forward and backward movement information and the rotation information associated with operation or movement of the linear member 11 to be described later that are received by the communication unit 125 from the operation device 102, the operation of the movement mechanism 110. More specifically, the control unit 127 controls, based on the forward and backward movement information and the rotation information described above, rotary driving of the rotatable bodies 122a, 123a, and 124 of the movement mechanism 110. Accordingly, forward and backward movements and rotation of the elongated body 200 are controlled.

The control unit 127 processes resistance information related to the load resistance received by the elongated body 200 and detected by the load sensor 112 to be described later. Specifically, the control unit 127 controls an operation of the communication unit 125 to transmit the resistance information received from the load sensor 112 to the operation device 102.

The control unit 127 includes a processing unit implemented by a processor such as a central processing unit (CPU) or a micro-processing unit (MPU). The processing unit executes a program stored in the storage unit 126 to operate each unit of the medical device 101. The control unit 127 according to the present embodiment may include a storage unit such as a read only memory (ROM) or a random access memory (RAM) in addition to or instead of the above-described storage unit 126.

The load sensor 112 can detect resistance information related to a load resistance received by the elongated body 200 held by the movement mechanism 110 in a blood vessel. The load sensor 112 according to the present embodiment can detect, as the resistance information, both information related to a load resistance received by the elongated body 200 in the longitudinal direction A and information related to a load resistance received by the elongated body 200 in a circumferential direction B around the central axis.

<Operation Device 102>

As described above, the operation device 102 can remotely operate the medical device 101 based on an operation of an operator such as a health care worker.

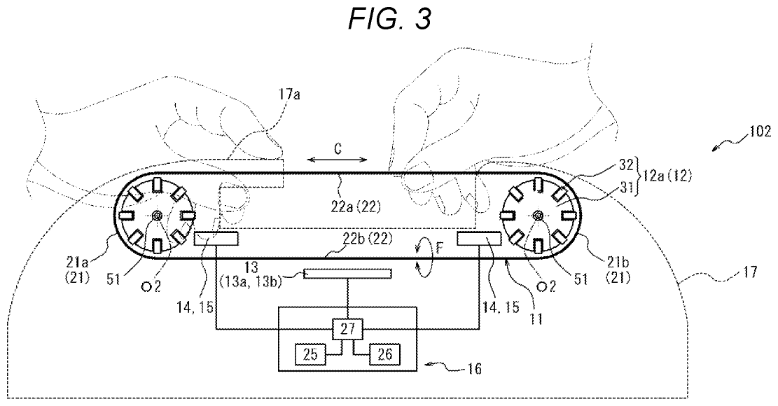

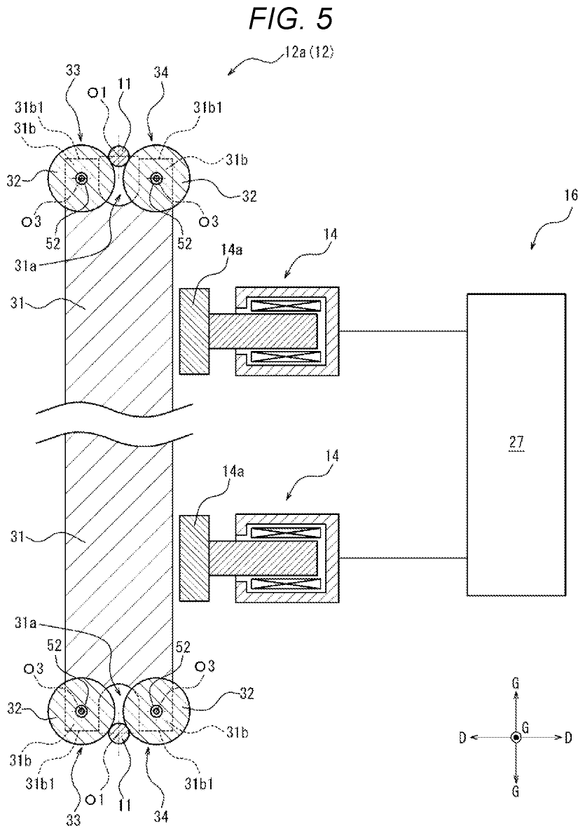

FIG. 3 is a diagram illustrating an example of the operation device 102. As shown in FIGS. 1 and 3, the operation device 102 according to the present embodiment includes an endless linear member 11. The operation device 102 can operate the medical device 101 based on a forward and backward movement of the linear member 11 in an endless axis direction C along an endless axis O1 (see FIG. 5). Accordingly, using the linear member 11 similar to using an actual elongated body 200 as a portion to be operated by the health care worker in the operation device 102, the operability for the health care worker can be improved. Further, by forming the linear member 11 in an endless shape, it is easy to achieve miniaturization of the operation device 102.

Hereinafter, further details of the operation device 102 according to the present embodiment will be described.

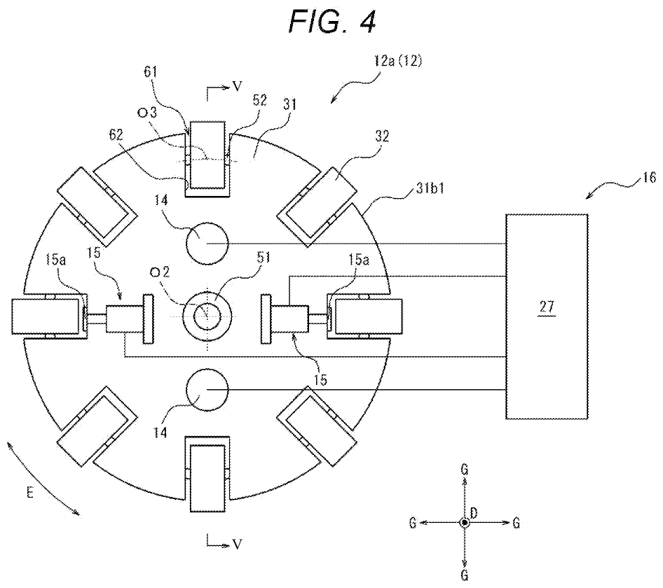

As shown in FIG. 3, the operation device 102 according to the present embodiment includes the above-described linear member 11, a plurality of support members 12, a movement detection sensor 13, variable resistance mechanisms (resistance adjustment mechanisms) 14, variable bearing resistance mechanisms (bearing resistance adjustment mechanisms) 15, a control device 16, and a housing 17. In FIG. 3, details of the variable resistance mechanisms 14 and the variable bearing resistance mechanisms 15 are omitted. FIG. 4 is a diagram illustrating details of the variable resistance mechanisms 14 and the variable bearing resistance mechanisms 15 that act on one support member 12. FIG. 5 is a cross-sectional view taken along the section line V-V in FIG. 4.

As shown in FIG. 3, the endless linear member 11 is wound around a plurality of support members 12 to be described later. In FIG. 3, for convenience of description, the linear member 11 and the support member 12 are illustrated in a non-contact state, but the linear member 11 and the support member 12 are actually in contact with each other. A cross-sectional outer shape of the linear member 11 orthogonal to the endless axis O1 is a substantially circular shape. The linear member 11 may be a tubular body that defines a hollow portion, or may be a solid body that is devoid of a hollow portion. An outer diameter of the linear member 11 may be, for example, 1 mm to 10 mm. Therefore, a feeling of a health care worker gripping the linear member 11 similar to a feeling of gripping the actual elongated body 200 such as a guide wire or a catheter can be achieved.

Constituent materials that may be used to form the linear member 11 are not particularly limited, and for example, a superelastic alloy such as a Ni—Ti alloy, stainless steel, or a cobalt-based alloy can be used. When these constituent materials are used, a linear member 11 having good flexibility and torque transmission performance can be easily implemented.

As shown in FIG. 3, the linear member 11 according to the present embodiment is stretched between two support members 12 to that the linear member 12 has an overall oval shape. More specifically, the linear member 11 according to the present embodiment includes a curved portion 21 wound around the support members 12, and a linear portion 22 extending linearly between the plurality of support members 12. The curved portion 21 according to the present embodiment includes a first curved portion 21a wound around one support member 12 of the two support members 12 and a second curved portion 21b wound around the other support member 12. The linear portion 22 according to the present embodiment includes a first linear portion 22a continuous with one end of the first curved portion 21a and one end of the second curved portion 21b, and a second straight portion 22b continuous with the other end of the first curved portion 21a and the other end of the second curved portion 21b.

The shape of the linear member 11 is not limited to a configuration including the curved portion 21 and the linear portion 22. However, the linear member 11 includes the linear portion 22, so that a portion gripped by a health care worker during operation can be implemented by the linear portion 22. In this way, moving operability of the linear member 11 in the endless axis direction C for the health care worker can be further improved.

The endless linear member 11 is wound around the plurality of support members 12. The plurality of support members 12 support the linear member 11. By supporting the linear member 11 by the plurality of support members 12, even when the linear member 11 is moved in the endless axis direction C, the shape of the linear member 11 is not likely to be deformed, and the operability for the operator can be improved.

The linear member 11 according to the present embodiment is stretched by the plurality of support members 12. Specifically, the two support members 12 according to the present embodiment abut against the linear member 11 on an inner side of the linear member 11 in a state of pressing an inner surface of the linear member 11. Therefore, the linear member 11 is supported by the two support members 12 and is stretched by the two support members 12. In this way, by stretching the linear member 11 by the plurality of support members 12, the shape of the linear member 11 moving in the endless axis direction C can be maintained. Therefore, the operability for the operator can be further improved.

More specifically, the two support members 12 according to the present embodiment are rotatable members 12a that each rotate around a respective central axis O2 according to the forward and backward movement of the linear member 11 in the endless axis direction C. Here, a rotational resistance of each rotatable member 12a around its central axis O2 according to the present embodiment is smaller than a frictional resistance between the linear member 11 and the rotatable members 12a. Therefore, when an operator such as a health care worker operates the linear member 11 to move the linear member 11 in the endless axis direction C, the rotatable members 12a can co-rotate with the linear member 11 by the frictional resistance between the linear member 11 and the rotatable members 12a. Therefore, for the operator, the linear member 11 can be easily moved in the endless axis direction C and the operability can be further improved, as compared with a case in which the support members 12 do not rotating.

In the present embodiment, all (two) of the support members 12 are rotatable members, but the present invention is not limited to this configuration. A part of the plurality of support members 12 may be implemented as the rotatable members 12a. However, as in the present embodiment, when all of the support members 12 are implemented by the rotatable members 12a, the operability for the operator can be further improved.

The operation device 102 according to the present embodiment includes only two support members 12. However, the operation device 102 is not limited to this configuration, and may be an operation device including three or more support members 12. When there are three or more support members 12, at least two support members 12 may be disposed on the inner side of the endless linear member 11. That is, the operation device 102 may include a support member 12 that abuts against an outer surface of the linear member 11 and applies tension to the linear member 11.

The rotatable members 12a serving as the support members 12 according to the present embodiment are rotatably supported by a shaft member 51 fixed to the housing 17 to be described later.

More specifically, each of the rotatable members 12a serving as the support members 12 according to the present embodiment includes a rotatable main body 31 and a plurality of rotatable bearing bodies 32. The rotatable main body 31 rotates around the central axis O2. The plurality of rotatable bearing bodies 32 are attached to an outer surface of the rotatable main body 31 around the central axis O2 so that the rotatable bearing bodies 32 are positioned radially outwardly of the central axis O2. The plurality of rotatable bearing bodies 32 rotate according to movement of the linear member 11 around the endless axis O1 while being in contact with the linear member 11.

As shown in FIGS. 4 and 5, the rotatable main body 31 according to the present embodiment is a disk-shaped rotatable body that is rotatably supported by the shaft member 51 constituting the central axis O2. That is, the rotatable main body 31 according to the present embodiment rotates around the shaft member 51. As shown in FIG. 5, an annular groove 31a is formed on a radially outward outer end surface of the disk-shaped rotatable main body 31 according to the present embodiment. In other words, an outer edge portion of the disk-shaped rotatable main body 31 according to the present embodiment includes two side plate portions 31b facing each other with a gap therebetween in a central axis direction D along the central axis O2. The above-described annular groove 31a is defined between the two side plate portions 31b. That is, the outer end surface of the disk-shaped rotatable main body 31 according to the present embodiment includes outer end surfaces 31b1 of the two facing side plate portions 31b. As shown in FIG. 4, the outer end surface 31b1 of each side plate portion 31b is formed with concave portions or recesses (grooves) 61 that are recessed radially inwardly at predetermined intervals in a circumferential direction E around the central axis O2. As shown in FIGS. 4 and 5, the shaft member 52 serving as the central axis O3 of the rotatable bearing body 32 to be described later is disposed between, of each side plate portion 31b, side surfaces 62 on both sides in the circumferential direction E that define the concave portions 61.

As shown in FIG. 4, eight concave portions 61 are formed in the outer end surface 31b1 of each side plate portion 31b according to the present embodiment at regular intervals in the circumferential direction E, but a distance between adjacent concave portions 61 and the number of the concave portions 61 are not particularly limited.

As described above, the plurality of rotatable bearing bodies 32 are attached to the outer surface of the rotatable main body 31 around the central axis O2. The plurality of rotatable bearing bodies 32 form a first rotatable bearing body group 33 and a second rotatable bearing body group 34 that are disposed at different positions in the central axis direction D at intervals around the central axis O2. The first rotatable bearing body group 33 according to the present embodiment is implemented by the rotatable bearing body 32 disposed in the concave portion 61 of one side plate portion 31b of the two side plate portions 31b. The second rotatable bearing body group 34 according to the present embodiment is implemented by the rotatable bearing body 32 disposed in the concave portion 61 of the other side plate portion 31b of the two side plate portions 31b. As shown in FIG. 5, the rotatable bearing bodies 32 constituting the first and second rotatable bearing body groups 33, 34 are arranged in side-by-side pairs (i.e., a pair is formed by one of the rotatable bearing bodies 32 in the first rotatable bearing body group 33 and one of the rotatable bearing bodies 32 in the second rotatable bearing body group 34 arranged side-by-side).

The linear member 11 according to the present embodiment is in contact with and supported by the rotatable bearing bodies 32 belonging to the first rotatable bearing body group 33 and the rotatable bearing bodies 32 belonging to the second bearing rotatable body group 34 at a position between the first rotatable bearing body group 33 and the second rotatable bearing body group 34 in the central axis direction D. More specifically, each rotatable bearing body 32 is rotatably supported by the respective shaft member 52 in one of the concave portions 61 of the side plate portion 31b of the above-described rotatable main body 31. The rotatable bearing body 32 can rotate around the shaft member 52. At least a part of the rotatable bearing body 32 enters or is positioned in the annular groove 31a between the two facing side plate portions 31b in a state in which the rotatable bearing body 32 is rotatably supported by the shaft member 52. That is, the rotatable bearing body 32 that is rotatably supported in the concave portion 61 of one side plate portion 31b and the rotatable bearing body 32 that is rotatably supported in the concave portion 61 of the other side plate portion 31b both enter or are positioned in the annular groove 31a. As shown in FIG. 5, the linear member 11 is in contact with and supported by both of the rotatable bearing bodies 32 in a manner straddling the rotatable bearing body 32 that is rotatably supported by the one side plate portion 31b and the rotatable bearing body 32 that is rotatably supported by the other side plate portion 31b.

As described above, the rotatable member 12a includes the plurality of rotatable bearing bodies 32, so that the linear member 11 is easily rotated in a rotational direction F around the endless axis O1. That is, the elongated body 200 held by the medical device 101 can be easily rotated around the central axis. Here, a rotational resistance of the rotatable bearing body 32 around the central axis O3 is smaller than a frictional resistance between the linear member 11 and the rotatable bearing body 32. As described above, the rotatable member 12a includes the rotatable bearing body 32, so that the linear member 11 can be easily rotated around the endless axis O1 and the operability for the operator can be improved, as compared with a configuration in which the rotatable member 12a does not include the rotatable bearing body 32.

Here, a relative positional relationship between the concave portion 61 of one side plate portion 31b of the rotatable main body 31 and the concave portion 61 of the other side plate portion 31b of the rotatable main body 31 in the circumferential direction E is not particularly limited. In the present embodiment, positions of the concave portions 61 in the two side plate portions 31b in the circumferential direction E substantially coincide with each other. In other words, the concave portions 61 in the two side plate portions 31b are arranged in a line in the central axis direction D. In this way, the linear member 11 is supported in a manner of being sandwiched between the rotatable bearing bodies 32 belonging to the first rotatable bearing body group 33 and the rotatable bearing bodies 32 belonging to the second rotatable bearing body group 34 at substantially the same positions in the circumferential direction E. Therefore, the linear member 11 is likely to be linearly extended and is not likely to undulate along the endless axis O1, and the linear member 11 is easily rotated around the endless axis O1. In contrast, in the circumferential direction E, a position where the rotatable bearing bodies 32 belonging to the first rotatable bearing body group 33 come into contact with the linear member 11 may be different from a position where the rotatable bearing bodies 32 belonging to the second rotatable bearing body group 34 come into contact with the linear member 11. In this way, the linear member 11 is likely to undulate along the endless axis O1. In this way, the linear member 11 is caught by the rotatable bearing body 32, and a slip between the linear member 11 and the rotatable member 12a can be prevented.

The movement detection sensor 13 according to the present embodiment can detect the forward and backward movement of the linear member 11 in the endless axis direction C. The movement detection sensor 13 according to the present embodiment can detect rotation of the linear member 11 in the axial rotational direction F. That is, the movement detection sensor 13 according to the present embodiment serves as both a forward and backward movement detection sensor 13a that detects a forward and backward movement of the linear member 11 in the endless axis direction C and a rotation detection sensor 13b that detects rotation of the linear member 11 in the axial rotational direction F. Such a movement detection sensor 13 may be, for example, an optical sensor including a light emitting portion that irradiates the linear member 11 with light and a light receiving portion that receives light reflected from the linear member 11. The movement detection sensor 13 can detect a movement direction and a movement amount of the linear member 11 based on a change in light received by the light receiving portion. However, the movement detection sensor 13 is not limited to such an optical sensor, and is not particularly limited as long as the movement detection sensor 13 has a configuration capable of detecting the forward and backward movement and the rotation of the linear member 11. The movement detection sensor 13 according to the present embodiment serves as both the forward and backward movement detection sensor 13a and the rotation detection sensor 13b, but the forward and backward movement detection sensor 13a and the rotation detection sensor 13b may be provided separately.

The movement detection sensor 13 according to the present embodiment detects the forward and backward movement of the linear member 11 itself, and may detect, for example, the rotation of the rotatable main body 31 of the rotatable member 12a. The movement detection sensor 13 according to the present embodiment detects the rotation of the linear member 11 itself, and may detect, for example, rotation of the rotatable bearing bodies 32 of the rotatable member 12a. However, as the movement detection sensor 13 according to the present embodiment, it is preferable to use a configuration that detects the movement of the linear member 11 itself. In this way, even when a slip occurs between the linear member 11 and the rotatable member 12a, the medical device 101 can be accurately controlled, and occurrence of an erroneous operation can be prevented.

As shown in FIG. 3, the movement detection sensor 13 according to the present embodiment detects the movement of the linear member 11 at a position of the linear portion 22 of the linear member 11, and may detect the movement of the linear member 11 at a position of the curved portion 21. However, as shown in FIG. 3, it is preferable that the movement detection sensor 13 detects the movement of the linear member 11 at a portion of the linear member 11 located in the housing 17. In this way, the light receiving portion can be prevented from receiving light from the surroundings and erroneously detecting the light.

The variable resistance mechanism 14 is capable of changing the rotation resistance of the rotatable member 12a around the central axis O2. The variable resistance mechanism 14 according to the present embodiment is controlled by a control device 16 to be described later. The control device 16 controls the variable resistance mechanism 14 based on resistance information related to a load resistance that is received by the elongated body 200 in the longitudinal direction A and that is detected by the load sensor 112 of the above-described medical device 101. Specifically, the control device 16 controls the variable resistance mechanism 14 such that the load resistance in the longitudinal direction A detected by the load sensor 112 acts on the linear member 11. Therefore, when the load sensor 112 detects a predetermined load resistance in the longitudinal direction A, the control device 16 controls the variable resistance mechanism 14 such that the same load resistance in the endless axis direction C is applied to the linear member 11.

As shown in FIGS. 4 and 5, for example, an electromagnetic solenoid can be used as the variable resistance mechanism 14. As shown in FIG. 5, the variable resistance mechanism 14 according to the present embodiment includes a movable portion 14a that is movable in the central axis direction D. In the central axis direction D, the movable portion 14a moves between a position where the movable portion 14a is in contact with a side surface of the rotatable main body 31 and a position where the movable portion 14a is not in contact with the side surface. Further, the movable portion 14a can change a pressing force for pressing the side surface of the rotatable main body 31 in a state in which the movable portion 14a is in contact with the side surface of the rotatable main body 31. In this way, by adjusting the contact state between the rotatable main body 31 and the movable portion 14a, the rotatable resistance of the rotatable main body 31 can be controlled. However, the variable resistance mechanism 14 is not particularly limited as long as the variable resistance mechanism 14 is capable of changing the rotation resistance of the rotatable member 12a around the central axis O2.

The variable bearing resistance mechanism 15 is capable of changing the rotation resistance of the rotation bearing body 32 with respect to the rotatable main body 31. The variable bearing resistance mechanism 15 according to the present embodiment is controlled by the control device 16 to be described later. The control device 16 controls the variable bearing resistance mechanism 15 based on resistance information related to a load resistance that is received by the elongated body 200 in the circumferential direction B around the central axis and that is detected by the load sensor 112 of the above-described medical device 101. Specifically, the control device 16 controls the variable bearing resistance mechanism 15 such that the load resistance in the circumferential direction B detected by the load sensor 112 acts on the linear member 11. Therefore, when the load sensor 112 detects a predetermined load resistance in the circumferential direction B, the control device 16 controls the variable bearing resistance mechanism 15 such that the same load resistance in the rotational direction F is applied to the linear member 11.

As shown in FIGS. 4 and 5, for example, an electromagnetic solenoid can be used as the variable bearing resistance mechanism 15. As shown in FIG. 4, the variable bearing resistance mechanism 15 according to the present embodiment includes a movable portion 15a that is movable in a radial direction G around the central axis O2 of the rotatable main body 31. In the radial direction G, the movable portion 15a moves between a position where the movable portion 15a is in contact with the rotatable bearing body 32 and a position where the movable portion 15a is not in contact with the rotatable bearing body 32. Further, the movable portion 15a can change a pressing force for pressing the rotatable bearing body 32 in a state in which the movable portion 15a is in contact with the rotatable bearing body 32. In this way, by adjusting the contact state between the rotatable bearing body 32 and the movable portion 15a, the rotation resistance of the rotatable bearing body 32 can be controlled. However, the variable bearing resistance mechanism 15 is not particularly limited as long as the variable bearing resistance mechanism 15 is capable of changing the rotational resistance of the rotatable bearing body 32 around the central axis O3.

The control device 16 transmits, to the medical device 101, forward and backward movement information related to a forward and backward movement of the linear member 11 in the endless axis direction C. The forward and backward movement is detected by the movement detection sensor 13 serving as the forward and backward movement detection sensor 13a. The control device 16 according to the present embodiment transmits, to the medical device 101, rotation information related to rotating movement of the linear member 11 in the axial rotational direction F. The rotating movement is detected by the movement detection sensor 13 serving as the rotatable detection sensor 13b. Further, the control device 16 controls operations of the variable resistance mechanism 14 and the variable bearing resistance mechanism 15. Specifically, the control device 16 controls the variable resistance mechanism 14 based on the load resistance received by the elongated body 200. The control device 16 controls the variable bearing resistance mechanism 15 based on the load resistance received by the elongated body 200. The load resistance received by the elongated body 200 is acquired based on resistance information related to the load resistance in the blood vessel that is received or sensed by the load sensor 112.

More specifically, the control device 16 according to the present embodiment includes the communication unit 25, a storage unit 26, and a control unit 27.

The communication unit 25 can communicate with the medical device 101 in a wired or wireless manner. The communication unit 25 is electrically connected to the communication unit 125 in the mechanism control device 111 of the medical device 101 by, for example, an electric signal line capable of transmitting and receiving an electric signal. The communication unit 25 can receive, from the medical device 101, the resistance information related to the load resistance received by the load sensor 112 in the blood vessel. In addition, the communication unit 25 can transmit the forward and backward movement information and the rotation information of the linear member 11 to the medical device 101.

The storage unit 26 stores a processing result of the control unit 27. The storage unit 26 may store various programs to be executed by the control unit 27. The storage unit 26 can be implemented by, for example, a random access memory (RAM), a read only memory (ROM), or the like.

The control unit 27 controls operations of the communication unit 25 and the storage unit 26. The control unit 27 processes various types of information received from the communication unit 25 and the storage unit 26.

Further, the forward and backward movement information and the rotation information of the linear member 11 detected by the movement detection sensor 13 are input to the control unit 27. The control unit 27 causes the communication unit 25 to transmit the forward and backward movement information and the rotation information to the medical device 101. The resistance information related to the load resistance received by the load sensor 112 of the medical device 101 in the blood vessel is input to the control unit 27 through the communication unit 25. The control unit 27 controls, based on this resistance information, operations of the movable portion 14a of the variable resistance mechanism 14 and the movable portion 15b of the variable bearing resistance mechanism 15.

The control unit 27 includes a processing unit implemented by a processor such as a central processing unit (CPU) or a micro-processing unit (MPU). The processing unit executes a program stored in the storage unit 26 to operate each unit of the operation device 102. The control unit 27 according to the present embodiment may include a storage unit such as a read only memory (ROM) or a random access memory (RAM) in addition to or instead of the above-described storage unit 26.

The housing 17 is an exterior member of the operation device 102. In FIG. 3, for convenience of description, the housing 17 is indicated by a broken line. The housing 17 covers a part of the linear member 11, the rotatable member 12a serving as the support member 12, the movement detection sensor 13, the variable resistance mechanism 14, the variable bearing resistance mechanism 15, and the control device 16.

As shown in FIG. 3, the first linear portion 22a, which is a part of the linear member 11 according to the present embodiment, is exposed to the outside of the housing 17. The operator can operate the linear member 11 by gripping the first linear portion 22a. The housing 17 includes a cylindrical portion 17a into which one end side of the first linear portion 22a of the linear member 11 is inserted. The cylindrical portion 17a is provided along the extending direction of the first linear portion 22a at a portion where the one end side of the first linear portion 22a of the linear member 11 exposed to the outside of the housing 17 enters the inside of the housing 17. By providing the cylindrical portion 17a in the housing 17, the operator can perform an operation by gripping the cylindrical portion 17a of the housing 17 with one hand and gripping the first linear portion 22a of the linear member 11 with the other hand. When inserting the elongated body 200 into a living body with hands, a health care worker usually performs the operation by gripping a connector portion at a proximal end of a tube such as a guiding catheter, which is inserted into the living body in advance, with one hand, and gripping the elongated body 200 with the other hand. Therefore, since the housing 17 includes the cylindrical portion 17a, an operator of the operation device 102, which is a health care worker, can operate the linear member 11 as in an actual medical procedure of inserting the elongated body 200 into a living body.

The detailed description above describes an operation device and the remote operation system representing an example of the inventive operation device and remote operation system disclosed here. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### REFERENCE SIGNS LIST

11: Linear member
12: Support member
12a: Rotatable member
13: Movement detection sensor
13a: Forward and backward movement detection sensor
13b: Rotatable detection sensor
14: Variable resistance mechanism
14a: Movable portion
15: Variable bearing resistance mechanism
15a: Movable portion
16: Control device
17: Housing
17a: Cylindrical portion
21: Curved portion
21a: First curved portion
21b: Second curved portion
22: Linear portion
22a: First linear portion
22b: Second linear portion
25: Communication unit
26: Storage unit
27: Control unit
31: Rotatable main body
31a: Annular groove
31b: Side plate portion
31b1: Side end surface
32: Rotatable bearing body
33: First rotatable bearing body group
34: Second rotatable bearing body group
51: Shaft member
52: Shaft member
55: Guide tube
55a: Connector
61: Concave portion
62: Side surface
100: Remote operation system
101: Medical device
102: Operation device
110: Movement mechanism
110a: Drive source
110b: Forward and backward moving mechanism
110c: Rotatable mechanism
111: Mechanism control device
112: Load sensor
121: Support main body
121a: Support surface
122: First rotatable body group
122a: Rotatable body belonging to first rotatable body group
123: Second rotatable body group
123a: Rotatable body belonging to second rotatable body group
124: Rotatable body
125: Communication unit
126: Storage unit
127: Control unit
200: Elongated body
A: Longitudinal direction of elongated body
B: Circumferential direction of elongated body
C: Endless axis direction of linear member
D: Central axis direction of rotatable member and rotatable main body
E: Circumferential direction of rotatable member and rotatable main body
F: Axial rotational direction of linear member
G: Radial direction of rotatable member and rotatable main body
T: Lesion area
O1: Endless axis of linear member
O2: Central axis of rotatable member and rotatable main body
O3: Central axis of rotatable bearing body

What is claimed is:

1. An operation device configured to remotely operate a movement mechanism that is configured to axially move an elongated medical body in a forward direction and in a backward direction while the elongated medical body is positioned in a living body, the operation device comprising:
an endless elongated member that includes a central axis and that is operable by a user to move the endless elongated member in one direction along the central axis of the endless elongated member and in an opposite direction along the central axis of the endless elongated member;
a movement detection sensor positioned and configured to detect the movement of the endless elongated member in the one direction and in the opposite direction; and
a control device that receives input from the movement detection sensor about the movement of the endless elongated member in the one direction and in the opposite direction and that is operatively connectable to the movement mechanism to control the movement mechanism and move the elongated medical body in the forward direction in response to the movement of the endless elongated member in the one direction as detected by the movement detection sensor and to move the elongated medical body in the backward direction in response to the movement of the endless elongated member in the opposite direction as detected by the movement detection sensor.

2. The operation device according to claim 1, wherein the movement mechanism to which the control device is operatively connectable is also configured to rotate the elongated medical body in a first rotational direction and in a second rotational direction while the elongated medical body is positioned in the living body, the endless elongated member being operable by the user to rotate the endless elongated member in one rotational direction about the central axis of the endless elongated member and in an opposite rotational direction about the central axis of the endless elongated member, the movement detection sensor being configured to detect rotational movement of the endless elongated member in the one rotational direction and in the opposite rotational direction, the control device being configured to: i) receive input from the movement detection sensor about the rotational movement of the endless elongated member in the one rotational direction and in the rotational opposite direction; and ii) control the movement mechanism to rotate the elongated medical body in the first rotational direction in response to the rotation of the endless elongated member in the one rotational direction as detected by the movement detection sensor and to rotate the elongated medical body in the second rotational direction in response to the rotation of the endless elongated member in the opposite rotational direction as detected by the movement detection sensor.

3. The operation device according to claim 1, wherein the endless elongated member is supported on at least two spaced apart rotatable support members so that the movement of the endless elongated member in the one direction along the central axis of the endless elongated member causes the at least two support members to rotate in a first rotational direction and the movement of the endless elongated member in the opposite direction along the central axis of the endless elongated member causes the at least two support members to rotate in a second rotational direction that is opposite the first rotational direction.

4. The operation device according to claim 3, wherein each rotatable support member includes a rotatable main body that is rotatable about a respective rotation axis, the rotation axis of each rotatable main body being parallel to one another, each rotatable support member also including a plurality of rotatable bearing bodies that are attached to the rotatable main body so that rotation of the rotatable main body about the rotation axis results in the plurality of rotatable bearing bodies rotating with the rotatable support member about the rotation axis.

5. The operation device according to claim 4, wherein the plurality of rotatable bearing bodies that are attached to a respective rotatable main body are rotatably attached to the respective rotatable main body so that each of the plurality of rotatable bearing bodies is rotatable relative to the rotatable main body about a respective rotation axis.

6. The operation device according to claim 5, wherein the plurality of rotatable bearing bodies are attached to the respective rotatable main body in side-by-side pairs, the endless elongated member being in contact with and positioned between an outer surface of the rotatable bearing bodies in a plurality of the side-by-side pairs of the rotatable bearing bodies.

7. The operation device according to claim 6, wherein the plurality of wherein the rotation axes of the rotatable bearing bodies constituting each side-by-side pair are parallel to one another.

8. The operation device according to claim 4, wherein some of the plurality of rotatable bearing bodies attached to each rotatable main body constitute a first rotatable bearing body group and others of the plurality of rotatable bearing bodies attached to each rotatable main body constitute a second rotatable bearing body group, the rotatable bearing bodies constituting the first rotatable bearing body group being circumferentially spaced apart from one another along an outer surface of the rotatable main body, the rotatable bearing bodies constituting the second rotatable bearing body group being circumferentially spaced apart from one another along the outer surface of the rotatable main body, the rotatable bearing bodies constituting the first rotatable bearing body group being spaced apart from the rotatable bearing bodies constituting the second rotatable bearing body group in a direction along the rotation axis of the rotatable main body.

9. The operation device according to claim 3, wherein each rotatable support member includes a rotatable main body that is rotatable about a respective rotation axis, the rotation axis of each rotatable main body being parallel to one another, and further comprising a variable resistance mechanism configured to change a rotation resistance of the rotatable member around the central axis, the control device being configured to control the variable resistance mechanism based on a load resistance received by the elongated medical body while the elongated medical body is positioned in the living body.

10. An operation device configured to remotely operate a medical device that includes a movement mechanism configured to move an elongated medical body that is insertable into a living body forward and backward along a longitudinal direction of the elongated medical body, the operation device comprising:
an endless linear member operatively connectable to the medical device and operable by a user to undergo forward and backward movement in an endless axial direction along an endless axis; and the elongated medical body being operated based on the forward and backward movement of the linear member in the endless axial direction along the endless axis.

11. The operation device according to claim 10, further comprising:
a forward and backward movement detection sensor configured to detect the forward and backward movement of the linear member in the endless axis direction; and
a control device configured to transmit, to the elongated medical body, forward and backward movement information related to the forward and backward movement of the linear member in the endless axis direction, the forward and backward movement being detected by the forward and backward movement detection sensor.

12. The operation device according to claim 11, further comprising a plurality of support members, the linear member being wound around the plurality of support members and being supported by the support members.

13. The operation device according to claim 12, wherein at least one of the plurality of support members is a rotatable member that rotates around a central axis during the forward and backward movement of the linear member in the endless axial direction.

14. The operation device according to claim 13, further comprising:
a variable resistance mechanism configured to change a rotation resistance of the rotatable member around the central axis, the control device being configured to control the variable resistance mechanism based on a load resistance received by the elongated medical body while the elongated medical body is positioned in the living body.

15. The operation device according to claim 13, wherein
the rotatable member includes:
a rotatable main body that rotates around the central axis; and
a plurality of rotatable bearing bodies that are attached to the rotatable main body to rotate together with the rotatable main body, the plurality of rotatable bearing bodies being spaced apart along an outer surface of the rotatable main body, the plurality of rotatable bearing bodies being rotatable in response to rotational movement of the linear member around the endless axis while the plurality of rotatable bearing bodies are in contact with the linear member,
some of the plurality of rotatable bearing bodies constituting a first rotatable bearing body group and others of the plurality of rotatable bearing bodies constituting a second rotatable bearing body group, the rotatable bearing bodies constituting the first rotatable bearing body group being circumferentially spaced apart from one another along the outer surface of the rotatable main body, the rotatable bearing bodies constituting the second rotatable bearing body group being circumferentially spaced apart from one another along the outer surface of the rotatable main body, the rotatable bearing bodies constituting the first rotatable bearing body group being spaced apart from the rotatable bearing bodies constituting the second rotatable bearing body group in a direction along the central axis, and
the linear member being in contact with and supported by at least some of the rotatable bearing bodies constituting the first rotatable bearing body group and at least some of the rotatable bearing bodies constituting the second rotatable bearing body group at a position between the first rotatable bearing body group and the second rotatable bearing body group in the direction along the central axis.

16. The operation device according to claim 15, further comprising:
a variable bearing resistance mechanism configured to change a rotation resistance of the rotatable bearing body with respect to the rotatable main body,
the control device being configured to control the variable bearing resistance mechanism based on a load resistance received by the elongated medical body while the elongated medical body is located in the living body.

17. The operation device according to claim 11, wherein
the movement mechanism is configured to rotate the elongated body around a central axis of the elongated medical body,
further comprising a rotation detection sensor configured to detect rotation of the linear member in an axial rotational direction around the endless axis, and
the control device being configured to transmit, to the medical device, rotation information detected by the rotation detection sensor about rotation of the linear member in the axial rotational direction.

18. A remote operation system for an elongated body, the remote operation system comprising:
the operation device according to claim 10, and
the medical device that is operatively connected to the operation device and remotely operated by the operation device.

19. A method comprising;
manually moving an endless elongated member that includes a central axis to move the endless elongated member in one direction along the central axis of the endless elongated member; and
operating a movement mechanism that is remotely located relative to the endless elongated member and that is operatively connected to an elongated medical device positioned in a living body based on the movement of the endless elongated member in the one direction along the central axis of the endless elongated member to advance the elongated medical device in the living body.

20. The method according to claim 19, further comprising rotating the endless elongated member about the central axis of the endless elongated member, and the operating of the movement mechanism including rotating the elongated medical device located in the living body based on the rotation of the endless elongated member about the central axis of the endless elongated member.
